(19) United States
(12) Patent Application Publication
(10) Pub. No.: US 2023/0355894 A1
(43) Pub. Date: Nov. 9, 2023

(54) MULTI-SLOT INJECTION NEEDLE

(71) Applicants: Mihir Umesh Gowda, Rocky Hill, CT (US); Simar Umesh Gowda, Rocky Hill, CT (US)

(72) Inventors: Mihir Umesh Gowda, Rocky Hill, CT (US); Simar Umesh Gowda, Rocky Hill, CT (US)

(21) Appl. No.: 18/312,233

(22) Filed: May 4, 2023

Related U.S. Application Data
(60) Provisional application No. 63/338,300, filed on May 4, 2022.

(51) Int. Cl. A61M 5/32

(52) U.S. Cl. CPC A61M 5/3291

(57) ABSTRACT

An injection needle including a shaft extending between a hub end, an injection end, and a lumen formed within the shaft. The lumen forms an opening that extends through the shaft from the hub end to the injection end. The injection end includes a bevel having a point and a heel, with the opening formed by the lumen passing through the bevel. Multiple slots may be formed within the shaft close to the injection end and near the bevel. The multiple slots may include a first group on one side of the shaft and a second group on an opposing side of the shaft.

### CROSS-REFERENCE TO RELATED APPLICATION

This application claims the benefit of priority of U.S. provisional application number 63/338,300, filed May 4, 2022, the contents of which are herein incorporated by reference.

### BACKGROUND OF THE INVENTION

The present invention relates to injection needles and, more particularly, to injection needles having multiple slots.

Needles have long been used in medical care because they allow for relatively quick administration of medications or aspiration of fluids for testing with relatively minimal invasion or damage to the human body. However, conventional needles have issues with the delivery or aspiration of fluids in patients.

Existing injection needles have one slot at the tip thus limiting the injection needles to administering or aspirating low viscosity fluids at slower flow rates. Thus, conventional needles require a slow and methodical injection of medicine or aspiration of fluids, leading to pain or increased perception of pain at the injection site. For higher flow volumes and rates or higher viscosity fluids, larger diameter needles must be used, leading to even more pain.

As can be seen, there is a need for an injection needle that allows for higher flow volumes and viscosities of fluids to be injected faster and with less pain than conventional technology allows. The present invention solves these problems by providing injection needles having multiple slots located towards a needle tip. The present invention thus facilitates injection or aspiration of larger fluid volumes and higher viscosity fluids in shorter periods of time, thereby minimizing pain experienced by the patient.

### SUMMARY OF THE INVENTION

In one aspect of the present invention, an injection needle comprises a shaft extending between a hub end and an injection end. The shaft further includes a lumen that extends within the full length of the shaft between the hub end and the injection end, such that a fluid or similar substance may pass through the shaft via the lumen either towards the hub end or towards the injection end. The shaft may comprise a metal, such as stainless steel or niobium, or other similarly strong and hard material suitable for piercing into or through at least a portion of a being or object.

In another aspect of the present invention, the hub end is configured to cooperate with a syringe or other like device that requires a needle for injecting a fluid or similar substance into a being or object and/or aspirating a fluid or similar substance out of a being or object.

In another aspect of the present invention, the injection end comprises at least a first bevel such that a point and heel are formed at the injection end of the shaft to allow the injection needle to pierce into or through at least a portion of a being or object.

In another aspect of the present invention, at least one slot is provided in the shaft proximal to the injection end. The at least one slot extends from an exterior surface of the shaft and into the lumen such that a fluid or similar material may pass through the at least one slot. The at least one slot may be located at any point around the circumference of the shaft. Further, the at least one slot may include a first slot and a second slot that are located on different sides of the shaft, such as in diametrically opposed positions, or in alternating positions on opposite sides of the shaft.

The present invention provides many advantages over the prior art. For example, it permits higher volumes of fluid to exit or enter the needle such that less time is needed to inject or aspirate a given volume of fluid relative to conventional needles.

These and other features, aspects and advantages of the present invention will become better understood with reference to the following drawings, description, and claims.

### BRIEF DESCRIPTION OF THE DRAWINGS

FIG. 1 is a perspective view of a syringe using a needle according to an embodiment of the present invention;

FIG. 2 is a perspective view of a needle according to an embodiment of the present invention;

FIG. 3 is a side view thereof;

FIG. 4 is a top view thereof;

FIG. 5 is a cross-sectional view thereof taken along line 5-5 in FIG. 3;

FIG. 6 is a detailed cross-sectional view of the tip thereof;

FIG. 7 is a perspective view of a needle according to an alternate embodiment of the present invention;

FIG. 8 is a side view thereof; and

FIG. 9 is a cross-sectional view thereof taken along line 9-9 in FIG. 8.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention an injection needle comprising a cylindrical shaft extending between a hub end and an injection end, wherein the shaft includes a lumen to allow fluid to pass through the shaft. The hub end is configured to cooperate with a syringe or other like device requiring a needle for the injection or aspiration of fluids or similar substances into or out of a being or object. The injection end comprises at least a first bevel including a point and heel such that the injection end may pierce into or through at least a portion of a being or object.

Further, at least one slot is provided in the shaft proximal to the injection end. The at least one slot provides an opening between an exterior surface of the shaft and the lumen such that a fluid or similar material may pass through the at least one slot. The at least one slot may be located at any point around the circumference of the shaft. Further, the at least one slot may include a first slot and a second slot that are located on the shaft in diametrically opposed positions.

It is noted that the materials of manufacture for the present invention are not particularly limited. The injection needle may comprise a metal, such as stainless steel or niobium, or other similarly strong and hard material, such as medical grade plastics, suitable for piercing into or through at least a portion of a being or object, with preference given to materials suitable for medical uses.

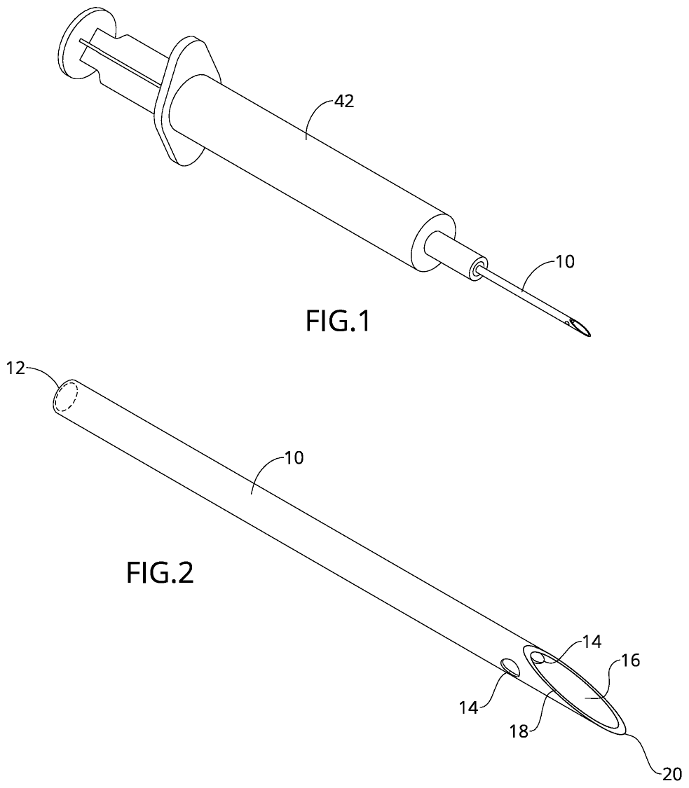

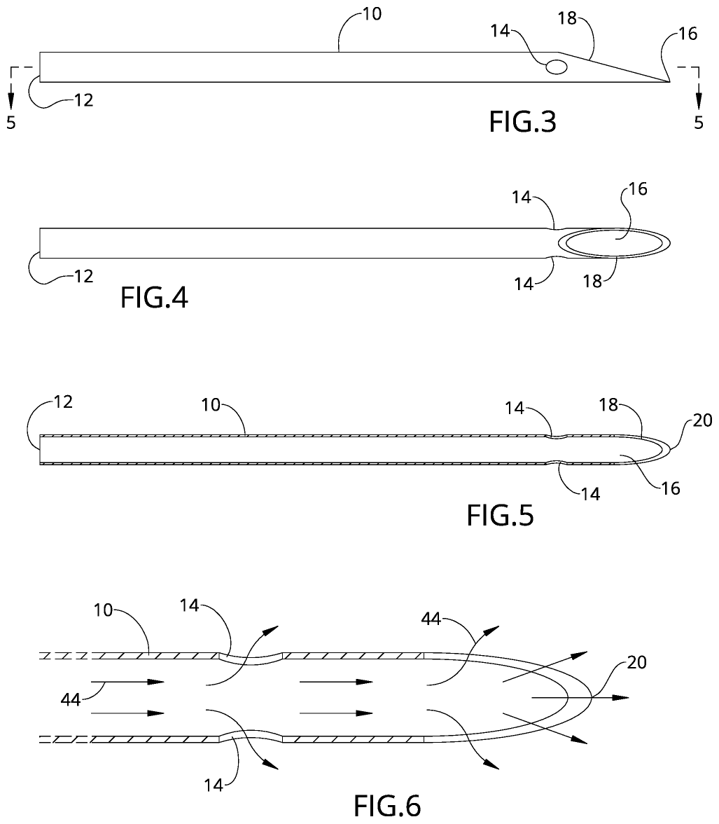

Referring now to FIGS. 1-9, embodiments of the present invention are shown.

In FIGS. 1-6, a first embodiment of an injection needle in accordance with the present invention is shown. As seen in FIG. 2, an injection needle according to the present invention may principally comprise a shaft 10, wherein the shaft 10 includes a hub end 12, an injection end comprising a bevel 18, and a lumen 16 passing between the hub end 12 and bevel 18 of the injection end for the full length of the shaft 10. Further, as seen in FIG. 1, the hub end 12 is configured to cooperate with and fasten to a syringe 42 or other like device that requires the use of a needle for the injection or aspiration of fluid 44 into or from a being or object. As shown in FIGS. 1-6, one embodiment of the shaft 10 is cylindrical. However, the shaft 10 may be formed in a variety of cross-sectional shapes that allow for piercing, e.g., circular, elliptical, polygonal.

The bevel 18 further includes a heel and a point 20 that are configured to allow the injection end to pierce into or through at least a portion of a being or object, e.g., through a person's skin and into the subcutaneous tissue, muscle or any targeted tissue. In one embodiment, the bevel 18 creates an elliptical primary opening into the lumen 16. The elliptical primary opening has a major and minor axis, with the major axis extending along a greater length of the shaft 10 than the minor axis. Additional embodiments of the present invention may have differently sized or shaped primary openings and may have additional bevels that when combined form the overall bevel 18.

As best seen in FIGS. 2-6, the injection needle further includes slots 14. These slots 14 may be formed on diametrically opposed sides of the shaft 10 proximal to the injection end. The slots 14 are preferably located on opposing sides of the shaft 10 that are orthogonal to the major axis of the elliptical primary opening formed by bevel 18. However, the slots 14 may be formed on other sides of the shaft 10. As shown, the slots 14 are located at least partially within the portion of the shaft 10 that includes the heel of bevel 18 within the circumference of the shaft 10. However, the slots 14 may be located on other portions of the shaft 10 that do not include the heel of bevel 18 within the circumference of the shaft 10. Further, while the slots 14 are shown to be elliptical, the slots 14 may be any size and shape suitable for the passage of fluid 44 into or out of the lumen 16.

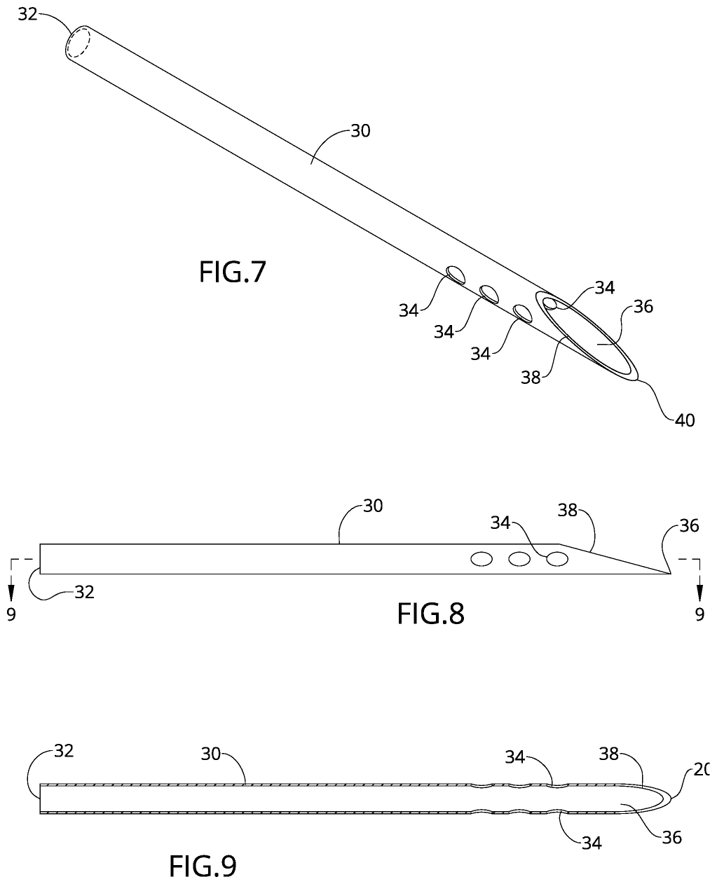

Referring now to FIGS. 7-9, an alternate embodiment of the injection needle of the present invention is shown. An injection needle comprises a shaft 30, wherein the shaft 30 includes a hub end 32, an injection end comprising a bevel 38, and a lumen 36 passing between the hub end 32 and bevel 38 of the injection end for the full length of the shaft 30. The bevel 38 further includes a heel and a point 40 that are configured to allow the injection end to pierce into or through at least a portion of a being or object, e.g., through a person's skin and into a target tissue.

The injection needle further includes slots 34. These slots 34 may be formed of a first group of slots 34 aligned on one side of the shaft 30 with a second group of corresponding slots 34 formed on diametrically opposed sides of the shaft 30 to the first group of slots 34. Alternatively, the slots 34 may be formed on opposing sides of the shaft 30 in an alternating pattern relative to one another.

The slots 34 are preferably located proximal to the injection end and bevel 38, and are further preferably formed on sides of the shaft 30 that are orthogonal to a major axis of an elliptical primary opening formed by the bevel 38 and lumen 36.

As shown, the slots 34 are sized and shaped equally and spaced at regular intervals along the shaft 30. However, the slots 34 may be formed shaped and sized differently from one another so long as they allow for fluid flows 44 to exit or enter the lumen 36 in the shaft 30. Further, the slots 34 may be spaced at irregular distances from one another so long as the slots 34 remain in a portion of the shaft 30 that is proximal to the injection end and that portion of the shaft 30 is entirely within a being or object when the injection needle is inserted into the being or object.

The present invention is thus an improvement on conventional injection needle technology. By providing multiple slots proximal to a tip of an injection needle, the present invention allows fluids to be injected or aspirated in a shorter time with less pain than conventional needles. Further, higher volume and higher viscosity fluids can be injected to or aspirated from a broader area in a shorter time using the present invention relative to conventional needles. The present invention also allows fluids to be delivered over larger tissue areas thereby aiding in uptake or dissemination of the delivered fluids in situations where quick uptake and dissemination is desirable, e.g., during the delivery of medication or anesthetic into a muscle during an intramuscular injection. Similarly, medications delivered as solutions or suspensions over larger tissue area can serve as a uniform depot for slow release of medications over time enabling a longer duration acting injectables that could enable less frequent injections for same therapeutic efficacy and better patient adherence to treatment. The present invention also allows for sampling of tissues from a larger tissue area during aspiration. Such larger sampling is useful in situations like when sampling a tumor.

The injection needle of the present invention may be used in conjunction with a variety of devices, such as with prefilled syringes for injections and self-injection devices, as a standalone needle, and used with catheters for aspiration or infusion.

It should be understood, of course, that the foregoing relates to exemplary embodiments of the invention and that modifications may be made without departing from the spirit and scope of the invention as set forth in the following claims.

What is claimed is:

1. An injection needle comprising a shaft extending between a hub end, an injection end, and a lumen forming an opening that extends through the shaft from the hub end to the injection end;
the injection end includes at least one bevel having a point and a heel, with the opening formed by the lumen passing through the bevel; and
at least one slot formed within the shaft proximal to the injection end.

2. The injection needle of claim 1, wherein the at least one slot includes a first slot and a second slot, with the first and second slots being located on different sides of the shaft.

3. The injection needle of claim 2, wherein the first and second slots are diametrically opposed.

4. The injection needle of claim 1, wherein the at least one slot is elliptical.

5. The injection needle of claim 1, wherein the at least one slot includes a first group of slots formed on one side of the shaft and a second group of slots formed on an opposing side of the shaft.

6. The injection needle of claim 5, wherein the first group of slots are aligned on the same side of the shaft, and the second group of slots are aligned on an opposing side of the shaft.

7. The injection needle of claim 5, wherein the first group of slots are spaced apart from one another at regular intervals, and the second group of slots are spaced apart from one another at regular intervals.

8. The injection needle of claim 5, wherein each of the first group of slots is diametrically opposed to a corresponding slot of the second group of slots.

9. The injection needle of claim 5, wherein the first group of slots and second group of slots are formed in an alternating pattern on opposing sides of the shaft.

10. The injection needle of claim 1, wherein the at least one slot is formed on the shaft at a location where a circumference of the shaft includes a portion of the bevel.
